(19) United States
(12) Patent Application Publication
(10) Pub. No.: US 2010/0070034 A1
(43) Pub. Date: Mar. 18, 2010

(54) CERVICAL INTRA-LAMINA FUSION IMPLANT DEVICE AND METHOD

(75) Inventors: Quentin Durward, North Sioux City, SD (US); Aaron D. Markworth, Saddle Brook, NJ (US)

Correspondence Address: Rahman LLC, 10025 Governor Warfield Parkway, Suite 110, Columbia, MD, 21044, US

(73) Assignee: Custom Spine, Inc., Parsippany, NJ (US)

(21) Appl. No.: 12/212,980

(22) Filed: Sep. 18, 2008

(51) Int. Cl. A61F 2/44; A61B 17/04

(52) U.S. Cl. 623/17.16; 623/17.11; 606/330

(57) ABSTRACT

A cervical intra-laminar fusion implant to be placed in cervical lamina includes a first lateral surface including a first arched edge, a second lateral surface positioned opposite to the first lateral surface, the second lateral surface including a plurality of posterior windows and a second arched edge, a top wall attached to the first lateral surface and the second lateral surface, the top wall having a plurality of teeth, a bottom wall positioned opposite to the top wall and attached to the first lateral surface and the second lateral surface, the bottom wall having a plurality of bottom teeth, a plurality of internal voids configured through the top wall and the bottom wall, and a connection mechanism connected to the top wall. The connection mechanism is positioned superior over the cervical lamina.

### BACKGROUND

1. Technical Field

The embodiments herein generally relate to medical devices, and, more particularly, to a cervical intra-lamina fusion device used during orthopedic surgeries.

2. Description of the Related Art

The human cervical spine begins at the base of the skull and consists of seven vertebrae with eight pairs of cervical nerves. The individual cervical vertebrae are abbreviated as C1, C2, C3, C4, C5, C6, and C7. The cervical nerves are also abbreviated as C1 through C8. C1 or Atlas is the topmost vertebra. C2 or Axis forms the pivot upon which C1 rotates. C1 along with C2 forms the joint connecting the skull and spine. Occiput is a bottom part of the skull where the spine meets the skull. The facets here are different than the rest of the spine. The occiput has convex facets which roll on the concave joint surfaces of C1.

Cervical spinal fusion surgeries are generally performed by removing a disc or a bone and fusing the vertebrae together with a bone graft either in front of or behind the spine (e.g., anterior and posterior cervical spinal fusion). The general procedure for the posterior cervical decompression includes a surgical approach or a disc approach. The posterior approach is often considered for large soft disc herniations that are lateral to (to the side of) the spinal cord. The disadvantage is that the disc space cannot be jacked open with a bone graft to give more space to the nerve root as it exits the spine. Also, since the posterior approach leaves most of the disc in place, there is a small chance (3% to 5%) that a disc herniation may recur in the future.

Posterior cervical decompression and fusion procedures are typically augmented with bone being inserted between the lamina and held in place with surgical cabling. The bone cannot be packed as well with graft or biologics to promote fusion, and does not conform to the anatomy for optimal load distribution. The cabling frequently subsides into the lamina bone. Furthermore, screws cannot fix the bone implant to the C2 cervical pedicle. None of the traditional standard interbody designs provide the surface contact and ideal placement on the cervical lamina with the ability to connect to adjacent anatomy. Most surgeons use bone and cabling which is complicated and can later subside causing additional instability.

### SUMMARY

In view of the foregoing, an embodiment herein provides a cervical intra-laminar fusion implant to be placed in cervical lamina and includes a first lateral surface including a first arched edge, a second lateral surface positioned opposite to the first lateral surface, the second lateral surface including a plurality of posterior windows and a second arched edge, a top wall attached to the first lateral surface and the second lateral surface, the top wall having a plurality of teeth, a bottom wall positioned opposite to the top wall and attached to the first lateral surface and the second lateral surface, the bottom wall having a plurality of bottom teeth, a plurality of internal voids configured through the top wall and the bottom wall, and a connection mechanism connected to the top wall. The connection mechanism may be positioned superior over the cervical lamina.

A pair of symmetrically contoured flanges may be attached to the connection mechanism. The flanges are preferably bendable. The connection mechanism may include a plurality of hooks. The connection mechanism may include a plate including a plurality of holes. The holes preferably accommodate securing means to attach the implant to an occipital plateau. The pair of symmetrically contoured flanges may include at least one hole that accommodates at least one securing means to be inserted through the lamina to provide fixation. The plurality of internal voids are preferably dimensioned and configured to be packed with bone graft material and the plurality of posterior windows are preferably dimensioned and configured for visibility and additional packing of bone graft material.

Another embodiment provides an apparatus providing optimal surface area to a lamina of a cervical spine and includes a first intra-laminar fusion implant having a first concave lateral surface including a first arched edge and a plurality of windows. The windows are preferably of substantially uniform length and width. A second concave lateral surface is preferably positioned opposite to the first lateral surface, the second concave lateral surface including a plurality of windows and a second arched edge. The first arched edge, the second arched edge, the first concave lateral surface, and the second concave lateral surface are preferably contoured to match an adjacent anatomy of the lamina of the cervical spine. A top wall is preferably attached to the first concave lateral surface and the second concave lateral surface, the top wall including a plurality of outwardly protruding top teeth, a bottom wall positioned opposite to the top wall and attached to the first lateral surface and the second lateral surface, the bottom wall including a plurality of outwardly protruding bottom teeth, a second intra laminar fusion implant positioned inferior to the first intra laminar fusion implant, a pair of symmetrically contoured flanges coupled to the bottom wall of the first intra laminar fusion implant, and a plate coupled perpendicular to the first intra laminar fusion implant. The plate is preferably at least one of a single midline design and a symmetrically double for bilateral fixation along sides of an occipital midline ridge plate.

A plurality of internal voids is preferably configured through the top wall and the bottom wall. A plurality of symmetrical hooks connected to the top wall. The plurality of hooks are preferably dimensioned and configured to be positioned over superior lamina. A pair of symmetrical arms may connect the first intra laminar fusion implant to the second intra laminar fusion implant. The pair of symmetrically contoured flanges are preferably bendable. The pair of symmetrically contoured flanges may include at least one hole that accommodates at least one securing means. The securing means may be inserted through the lamina to provide extra fixation. The plate may further include a plurality of holes that accommodate securing means to attach the implant to an occipital plateau for total occipto atlanto-axial fixation. The outwardly protruding top and bottom teeth may be dimensioned and configured to provide a mechanical interlock between the lamina and the apparatus.

Another embodiment provides a method to stabilize and improve immobilization of a human cervical spine by inserting a cervical intra-laminar implant using an inserter too. The method includes engaging the inserter tool with the cervical intra-laminar implant, the inserter tool including a shaft of an adequate length, the shaft includes a first end and a second end, the first end including a protrusion, the cervical intra-laminar implant including a first lateral surface including a first arched edge, a second lateral surface positioned opposite to the first lateral surface, the second lateral surface having a plurality of posterior windows and a second arched edge, a top wall attached to the first lateral surface and the second lateral surface, the top wall having a plurality of teeth, a bottom wall positioned opposite to the top wall and attached to the first lateral surface and the second lateral surface, the bottom wall having a plurality of bottom teeth, a plurality of internal voids configured through the top wall and the bottom wall, and a connection mechanism connected to the top wall, the connection mechanism is positioned superior over the cervical lamina. The method may further include attaching the protrusion to the cervical intra-laminar implant, the protrusion is attached via at least one of a thread, a snap fitting, and a press fit stud, packing bone graft material with the posterior windows between the plurality of internal voids, and impacting the cervical intra-laminar implant with a loaded the inserter tool. The cervical intra-laminar implant may be wedged between the human cervical spine.

The cervical intra-laminar implant may further include a pair of symmetrically contoured flanges attached to the connection mechanism. The flanges are preferably bendable. The connection mechanism may comprise a plurality of hooks. The connection mechanism may include a plate having a plurality of holes. The holes may accommodate securing means to attach the implant to an occipital plateau. The pair of symmetrically contoured flanges may include at least one hole that accommodates at least one securing means to be inserted through the lamina to provide fixation. The plurality of internal voids may be dimensioned and configured to be packed with bone graft material and the plurality of posterior windows may be dimensioned and configured for visibility and additional packing of bone graft material.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIGS. 1A and 1B illustrate perspective views of an intra-lamina fusion implant according to an embodiment herein;

FIGS. 2A and 2B illustrate a front view and a back view of the intra-lamina fusion implant of FIGS. 1A and 1B, respectively, fixed to two hooks according to an embodiment herein;

FIGS. 3A and 3B illustrate a front view and a back view of the intra-lamina fusion implant of FIGS. 2A and 2B, respectively, fixed to a second intra lamina fusion implant according to an embodiment herein;

FIGS. 4A and 4B illustrate a front view and a back view of the intra-lamina fusion implant of FIGS. 1A and 1B, respectively, fixed to two contoured flanges according to an embodiment herein;

FIGS. 5A and 5B illustrate a front view and a back view of the intra-lamina fusion implant of FIGS. 4A and 4B, respectively, fixed to a plate in place of the hooks according to an embodiment herein;

FIG. 6 illustrate a front view of the implant of FIGS. 1A and 1B inserted to a lamina according to an embodiment herein; and

FIG. 7 is a flow diagram illustrating a method to stabilize and improve immobilization of a human cervical spine by inserting a cervical intra-laminar implant of FIG. 1 using an inserter tool according to an embodiment herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

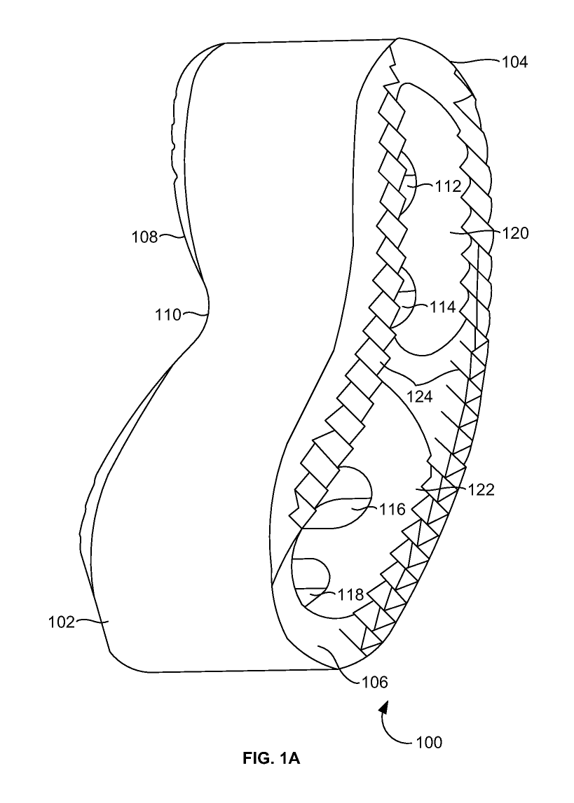

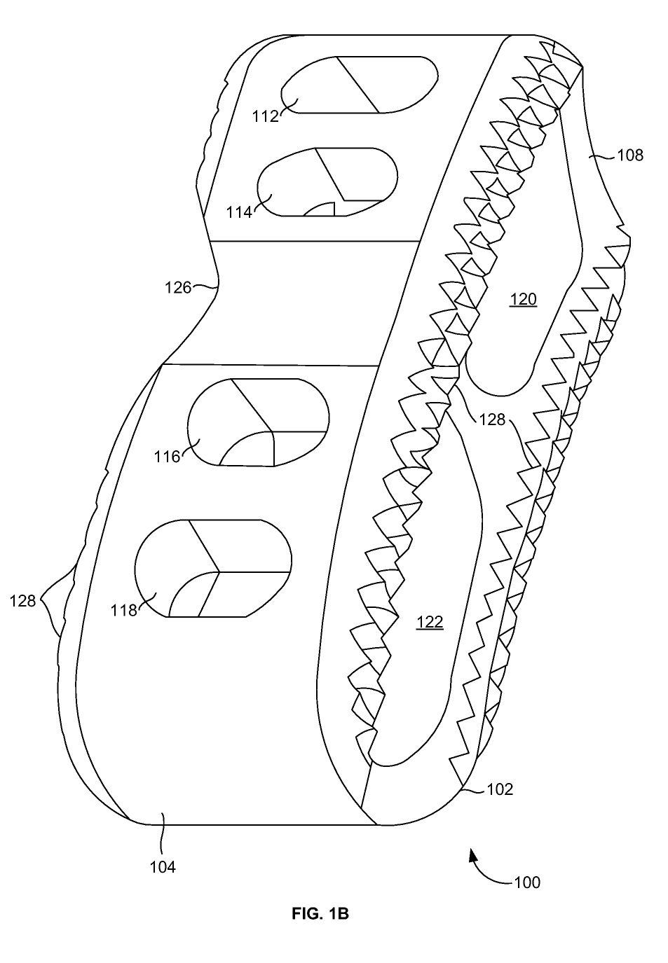

The embodiments herein provide an optimal surface coverage with the lamina improving immobilization of the cervical spine and reducing the chances of subsidence. Referring now to the drawings, and more particularly to FIGS. 1A through 7, where similar reference characters denote corresponding features consistently throughout the figures, there are shown preferred embodiments.

FIGS. 1A and 1B illustrate perspective views of an intra-lamina fusion implant 100 according to an embodiment herein. The implant 100 is a monolithic block which includes a first concave lateral surface 102, a second concave lateral surface 104, a top wall 106, and a bottom wall 108. The second concave surface 104 is positioned opposite to the first concave lateral surface 102. The top wall 106 and the bottom wall 108 are positioned opposite to each other and are attached to the first concave surface 102 and the second concave surface 104.

The implant 100 further includes a first arched edge 110 and a plurality of posterior windows 112, 114, 116, 118 of substantially the same length and width which are present on top wall 106. In addition, the implant 100 includes two internal voids 120, 122 which are configured through the top wall 106 and the bottom wall 108. The length and width of the two internal voids 120, 122 are almost equal. A plurality of outwardly protruding top teeth 124 is present on the top wall 106. The second concave lateral surface 104 further includes a second arched edge 126 and the bottom wall 108 further includes a plurality of outwardly protruding bottom teeth 128. The top teeth 124 are aligned with the bottom teeth 128.

The structure of the implant 100 is slightly oblique. The pair of concave surfaces 102, 104 are contoured to match with lamina anatomy of a human. The first arched edge concave edge 110 and the second arched edge 126 are textured to help fixation of the implant 100 with lamina (not shown). The internal voids 120, 122 are configured for packing bone graft material which helps in further bone formation. The posterior windows 112, 114, 116, 118 are configured for visibility and additional packing. The top teeth 124 and the bottom teeth 128 may help in penetrating the muscles of the lamina and provide a mechanical interlock between the implant 100 and the lamina. The mechanical stability afforded by the teeth 124, 128 may minimize the risk of postoperative expulsion of the implant 100.

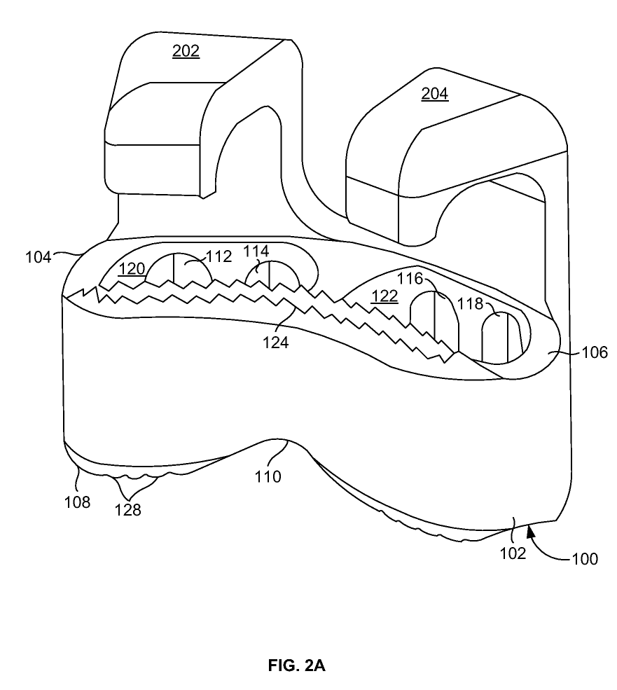

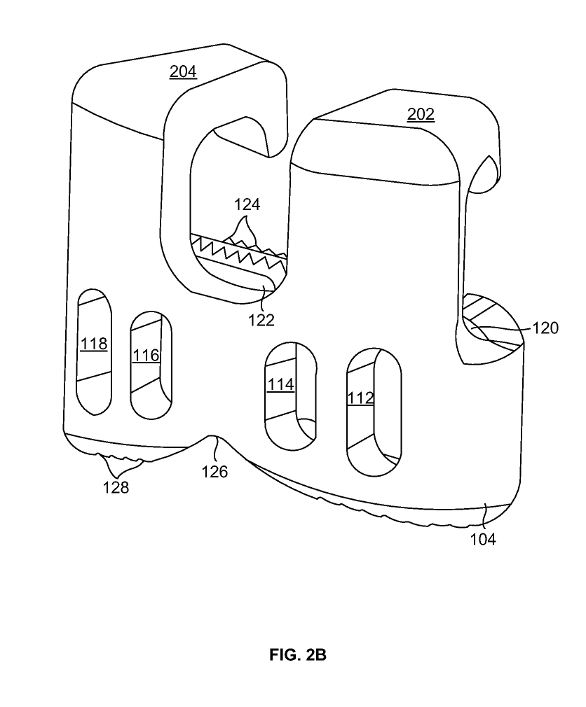

FIGS. 2A and 2B illustrate a front view and a back view of the intra-lamina fusion implant 100 of FIGS. 1A and 1B, respectively, fixed to two hooks 202, 204 according to an embodiment herein. In a preferred embodiment, the implant 100 further includes the two symmetrical hooks 202, 204 which are coupled to one side of the top wall 106 of the implant 100. The hooks 202, 204 are dimensioned and configured to fix properly and to go over the superior lamina. The hooks 202, 204 are either molded or machined as one piece, or are separate (can snap, screw, or slide on, etc.). The hook position may be fixed with respect to the implant 100 or compressible to pinch the lamina.

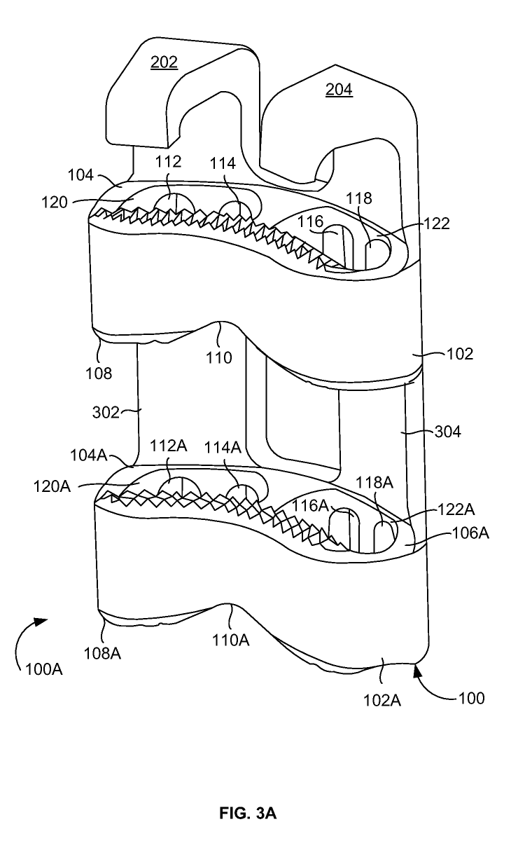

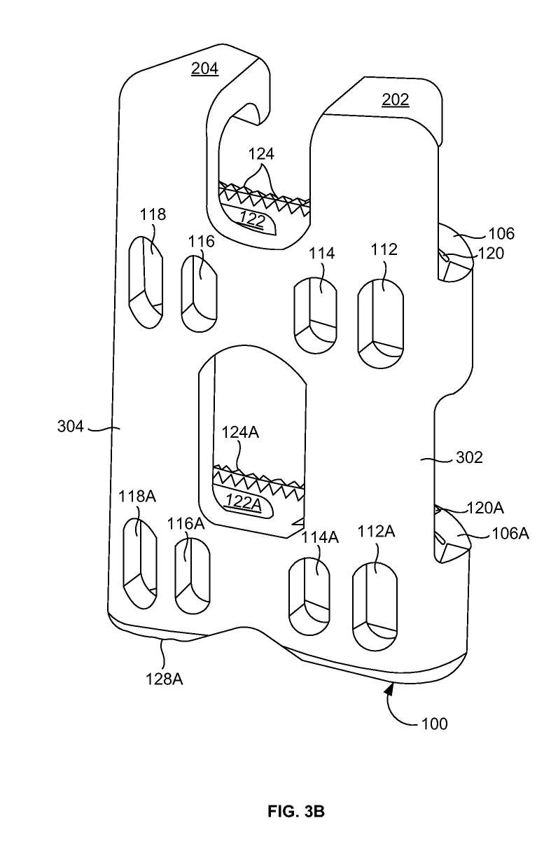

FIGS. 3A and 3B illustrate a front view and a back view of the intra-lamina fusion implant 100 of FIGS. 2A and 2B, respectively, fixed to a second intra lamina fusion implant according to an embodiment herein. In a preferred embodiment, the implant 100 further includes an intra-lamina fusion implant 100A for addressing the adjacent level. The intra-lamina fusion implant 100A is coupled to the implant 100 by a pair of arms 302, 304. The implant 100 and the intra-lamina fusion implant 100A are positioned in a parallel alignment to each other.

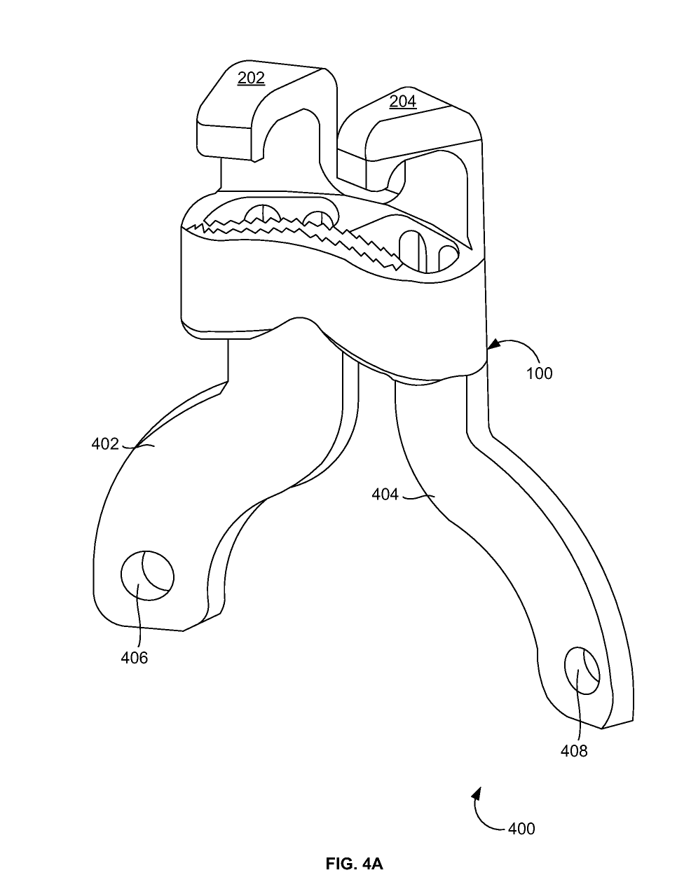

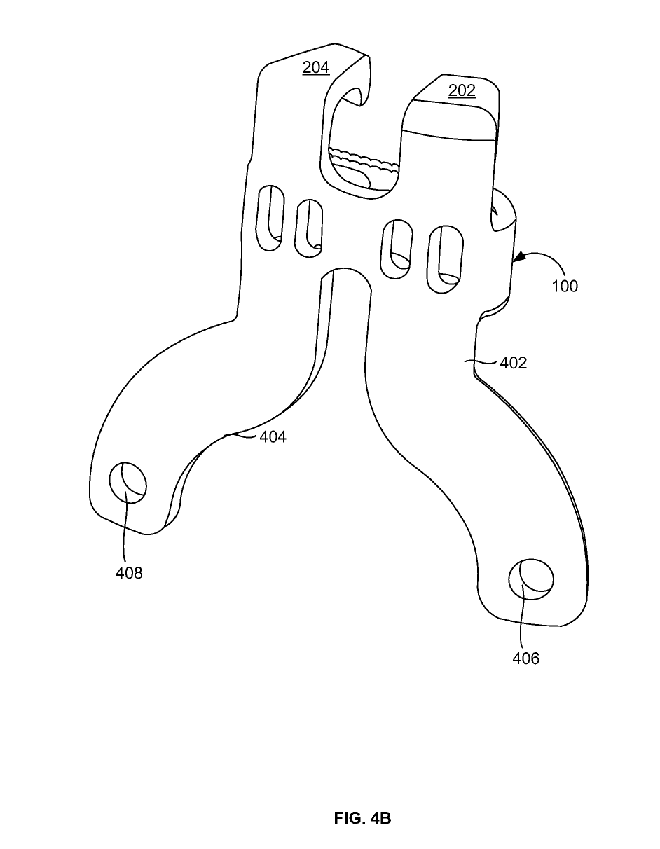

FIGS. 4A and 4B illustrate a front view and a back view of the intra-lamina fusion implant 100 of FIGS. 1A and 1B, respectively, fixed to two contoured flanges 402, 404 according to an embodiment herein. The symmetric contoured flanges 402, 404 may be bendable for targeting cervical pedicles, lateral masses, or transarticular screw placement. Each of the flanges 402, 404 further include a screw hole 406, 408. The screw holes 406, 408 are textured for accommodating screws (not shown) to be fixed into the lamina. This helps in extra fixation. The flanges 402, 404 may be the same or different material as the implant 100 and one-piece or attachable similar to the hooks 202, 204.

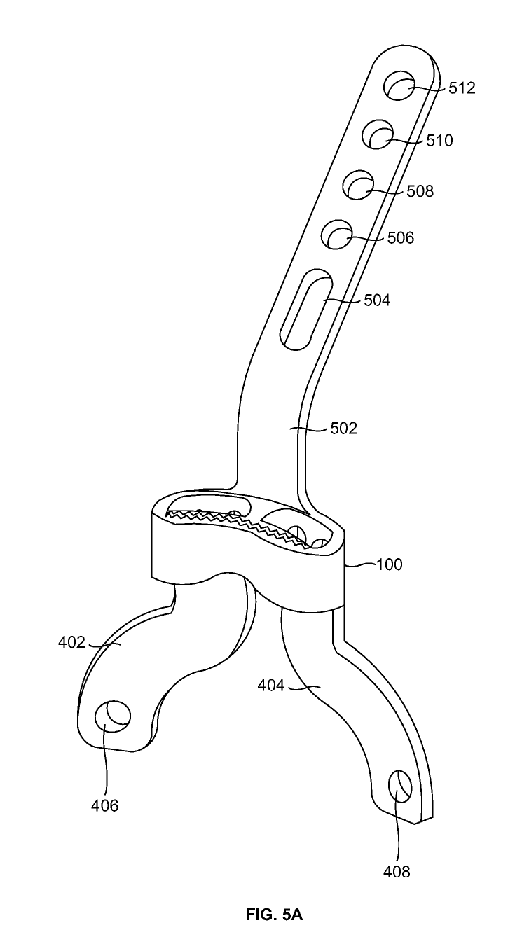

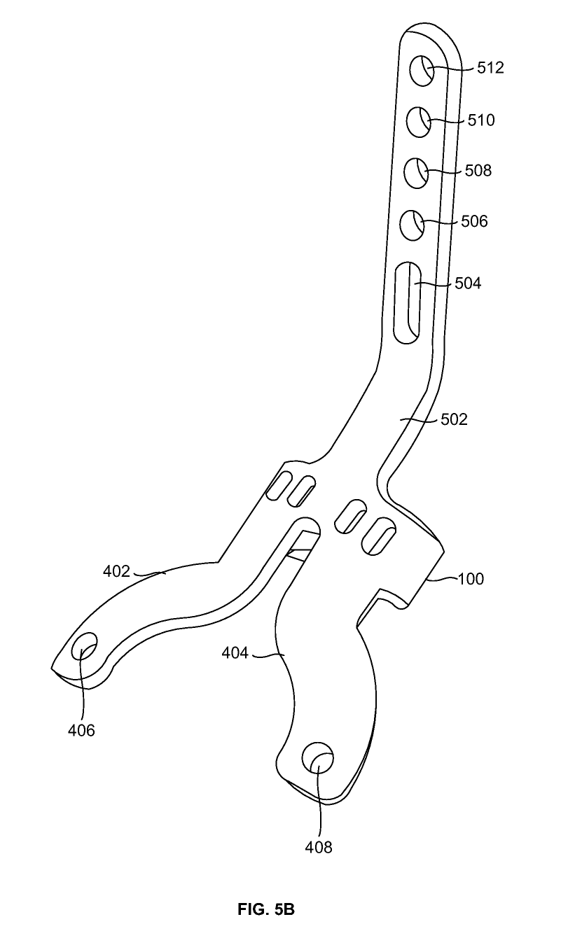

FIGS. 5A and 5B illustrate a front view and a back view of the intra-lamina fusion implant 100 of FIGS. 4, fixed to a plate 502 in place of the hooks 202, 204 according to an embodiment herein. The plate 502 further includes a plurality of screw holes 504, 506, 508, 510, 512 which are dimensioned and configured for accommodating screws (not shown) for attachment to the occipital plateau (not shown). The plate 502 may be a single midline design or symmetrically double for bilateral fixation along the sides of the occipital midline ridge. The plate 502 can have similar material properties as the flanges 402, 404 or hooks 202, 204.

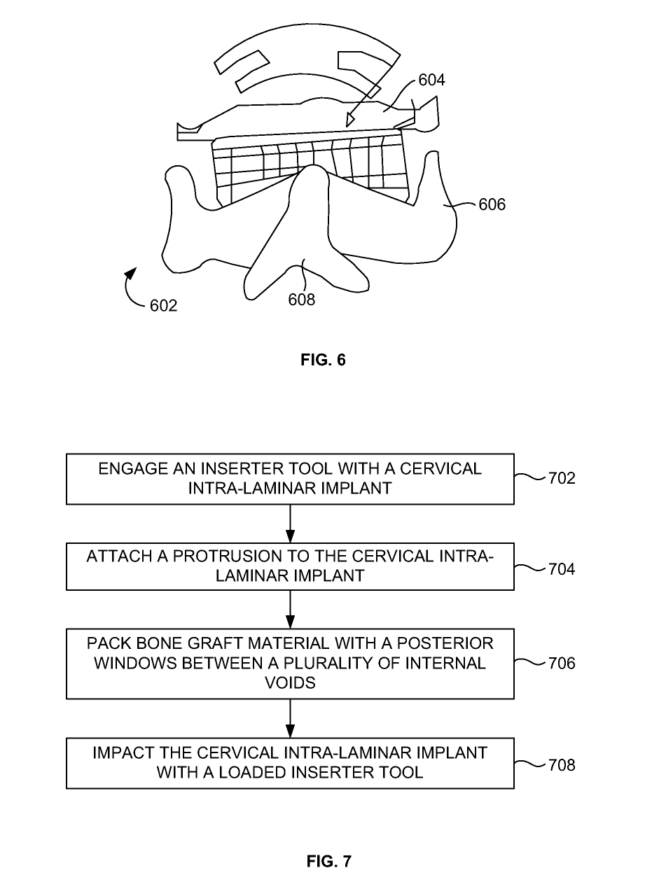

FIG. 6 illustrates a front view of the implant 100 inserted to a vertebrae 602 according to an embodiment herein. The vertebrae 602 include a C1 lamina 604, a C2 lamina 606, and a spinous process 608. The implant 100 is fixed in a space between the C1 lamina 604 and the C2 lamina 606. The arched edge 126 of the implant 100 matches with a curved structure of the spinous process 608.

The implant 100 would be ideally inserted or impacted by means of an inserter tool (not shown). In a preferred mode, this inserter may comprise of a shaft of an adequate length. On one end, there may be a protrusion that is attached to the implant 100 via a thread, a snap fitting or simply a press fit stud. On the opposite end, the inserter may have a handle with an impact surface. When the implant loaded inserter is lightly impacted, the implant 100 may ideally get wedged between cervical lamina to slightly decompress them and provide fusion surfaces. The inserter is positively connected to the implant not requiring pliers or forceps to provide compression on the implant. Furthermore, no cabling is required to be wound or threaded through the implant 100. The inserter may also be capable of activating the compression of the hooks 202, 204 or flanges 402, 404 or plate 502 if they are made in a two-piece configuration.

FIG. 7, with reference to FIGS. 1A through 6, is a flow diagram illustrating a method to stabilize and improve immobilization of a human cervical spine by inserting a cervical intra-laminar implant 100 of FIG. 1 using an inserter tool (not shown) according to an embodiment herein. In step 702, the inserter tool is engaged with the cervical intra-laminar implant. The inserter tool including a shaft of an adequate length. The shaft includes a first end and a second end. The first end includes a protrusion. In step 704, the protrusion is attached to the cervical intra-laminar implant 100. The protrusion is attached via at least one of a thread, a snap fitting, and a press fit stud. In step 706, bone graft material is packed with the posterior windows 112-118 between the plurality of internal voids 120-122. In step 708, the cervical intra-laminar implant 100 is impacted with a loaded inserter tool. The cervical intra-laminar implant 100 is wedged between the human cervical spine.

The implant 100 can be utilized in surgery to stabilize the human cervical spine. It may be used to replace or augment a cervical lamina to aid in stabilization or fusion of the cervical spine. In addition, the implant 100 can be used over multiple segments or attached to the occiput for total occipito-atlanto axial fixation. Ideally the implant 100 can be used with some form of the many available cervico-thoracic fixation devices from a posterior approach. The implant 100 is an improvement over existing devices in terms of implantation method, ease of use, and patient stability after implantation. It is designed to work with any appropriate implantable material and may utilize all the standard surgical tools that accompany such devices.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. A cervical intra-laminar fusion implant to be placed in cervical lamina, said implant comprising:
a first lateral surface comprising a first arched edge;
a second lateral surface positioned opposite to said first lateral surface, said second lateral surface comprising a plurality of posterior windows and a second arched edge;
a top wall attached to said first lateral surface and said second lateral surface, said top wall comprising a plurality of teeth;
a bottom wall positioned opposite to said top wall and attached to said first lateral surface and said second lateral surface, said bottom wall comprising a plurality of bottom teeth;
a plurality of internal voids configured through said top wall and said bottom wall; and
a connection mechanism connected to said top wall, wherein said connection mechanism is positioned superior over said cervical lamina.

2. The implant of claim 1, further comprising a pair of symmetrically contoured flanges attached to said connection mechanism, wherein said flanges are bendable.

3. The implant of claim 1, wherein said connection mechanism comprises a plurality of hooks.

4. The implant of claim 1, wherein said connection mechanism comprises a plate comprising a plurality of holes, wherein said plurality of holes accommodate securing means to attach said implant to an occipital plateau.

5. The implant of claim 2, wherein said pair of symmetrically contoured flanges comprises at least one hole that accommodates at least one securing means to be inserted through said lamina to provide fixation.

6. The implant of claim 1, wherein said plurality of internal voids are dimensioned and configured to be packed with bone graft material and said plurality of posterior windows are dimensioned and configured for visibility and additional packing of bone graft material.

7. An apparatus to provide optimal surface area to a lamina of a cervical spine, said apparatus comprising:
a first intra-laminar fusion implant comprising:
a first concave lateral surface comprising a first arched edge and a plurality of windows, wherein said plurality of windows are of substantially uniform length and width;
a second concave lateral surface positioned opposite to said first lateral surface, said second concave lateral surface comprising a plurality of windows and a second arched edge, wherein said first arched edge, said second arched edge, said first concave lateral surface, and said second concave lateral surface are contoured to match an adjacent anatomy of said lamina of said cervical spine;
a top wall attached to said first concave lateral surface and said second concave lateral surface, said top wall comprising a plurality of outwardly protruding top teeth;
a bottom wall positioned opposite to said top wall and attached to said first lateral surface and said second lateral surface, said bottom wall comprising a plurality of outwardly protruding bottom teeth;
a second intra laminar fusion implant positioned inferior to said first intra laminar fusion implant;
a pair of symmetrically contoured flanges coupled to said bottom wall of said first intra laminar fusion implant; and
a plate coupled perpendicular to said first intra laminar fusion implant, wherein said plate is at least one of a single midline design and a symmetrically double for bilateral fixation along sides of an occipital midline ridge plate.

8. The apparatus of claim 7, further comprising a plurality of internal voids configured through said top wall and said bottom wall.

9. The apparatus of claim 7, further comprising a plurality of symmetrical hooks connected to said top wall, wherein said plurality of hooks are dimensioned and configured to be positioned over superior lamina.

10. The apparatus of claim 7, further comprising a pair of symmetrical arms connecting said first intra laminar fusion implant to said second intra laminar fusion implant.

11. The apparatus of claim 7, wherein said pair of symmetrically contoured flanges are bendable.

12. The apparatus of claim 7, wherein said pair of symmetrically contoured flanges comprises at least one hole that accommodates at least one securing means, wherein said securing means is inserted through said lamina to provide extra fixation.

13. The apparatus of claim 7, wherein said plate further comprises a plurality of holes that accommodate securing means to attach said implant to an occipital plateau for total occipto atlanto-axial fixation.

14. The apparatus of claim 7, wherein said outwardly protruding top and bottom teeth are dimensioned and configured to provide a mechanical interlock between said lamina and said apparatus.

15. A method to stabilize and improve immobilization of a human cervical spine by inserting a cervical intra-laminar implant using an inserter tool, said method comprising:
engaging said inserter tool with said cervical intra-laminar implant, said inserter tool comprising a shaft of an adequate length, wherein said shaft comprises a first end and a second end, said first end comprising a protrusion, wherein said cervical intra-laminar implant comprising:
a first lateral surface comprising a first arched edge;
a second lateral surface positioned opposite to said first lateral surface, said second lateral surface comprising a plurality of posterior windows and a second arched edge;
a top wall attached to said first lateral surface and said second lateral surface, said top wall comprising a plurality of teeth;
a bottom wall positioned opposite to said top wall and attached to said first lateral surface and said second lateral surface, said bottom wall comprising a plurality of bottom teeth;
a plurality of internal voids configured through said top wall and said bottom wall; and
a connection mechanism connected to said top wall, wherein said connection mechanism is positioned superior over said cervical lamina;
attaching said protrusion to said cervical intra-laminar implant, wherein said protrusion is attached via at least one of a thread, a snap fitting, and a press fit stud;
packing bone graft material with said posterior windows between said plurality of internal voids; and
impacting said cervical intra-laminar implant with a loaded said inserter tool, wherein said cervical intra-laminar implant is wedged between said human cervical spine.

16. The method of claim 15, wherein said cervical intra-laminar implant further comprises a pair of symmetrically contoured flanges attached to said connection mechanism, wherein said flanges are bendable.

17. The method of claim 15, wherein said connection mechanism comprises a plurality of hooks.

18. The method of claim 15, wherein said connection mechanism comprises a plate comprising a plurality of holes, wherein said plurality of holes accommodate securing means to attach said implant to an occipital plateau.

19. The method of claim 16, wherein said pair of symmetrically contoured flanges comprises at least one hole that accommodates at least one securing means to be inserted through said lamina to provide fixation.

20. The method of claim 15, wherein said plurality of internal voids are dimensioned and configured to be packed with bone graft material and said plurality of posterior windows are dimensioned and configured for visibility and additional packing of bone graft material.
